SEQUENCE LISTING (6 sequences)

SEQ ID NO: 1 - DNA, 299 bp, HUMAN
  CDS (21)..(299)
gcaagagggagccacggccgatgacagaaaattcactttccgagatggcctctaaatcctggctgaattttttaaccttcctctatggatcggcaatagggtttattttattttctcagctacttagtattttgttgggagaagagggtgacacccagactaatgttcttcataatgatcctcatgcgaggcattcagatgataatggacagaatcatctaggaggacaaatgaacttcaatgcagattctagccaacgtaaagatgagaacacagaaatcgctgaaaacctctattag

SEQ ID NO: 2 - protein, 92 aa, HUMAN
MTENSLSEMASKSWLNFLTFLYGSAIGFILFSQLLSILLGEEGDTQTNVLHNDPHARHSDDNGQNHLGGQMNFNADSSQRKDENTEIAENLY

SEQ ID NO: 3 - DNA, 489 bp, HUMAN
  CDS (55)..(489)
catctaaaaagactgatgaagttgattgcaaatgctagtcatcataaataccagaacacaggtgtcactgacaaactctatcaaaagatgaaaattctttgctggattatgacaggacctcaaaatctagaaaaaaagatcagacgcatcagagatacatgggcccagggttgcaataaagcgttgtttatgagctcaaaagaaaataaagacttctctactgtgggattacacaccaaagaagacagaaaccaactgtcctggaaaatagttaaagcttttctatatgctcatgaccattatctggaatacatggattggttcatgaaagcagatgatgatatatgtatatatatcacattggacaacttgaaatggcttctcacaaactataaccctgatgaatccacttactttgggaaaagatttaagcactgcagaaaacaggactacatgactggaggagcaggatatgtactgagcaaagaa

SEQ ID NO: 4 - protein, 145 aa, HUMAN
NTGVTDKLYQKMKILCWIMTGPQNLEKKIRRIRDTWAQGCNKALFMSSKENKDFSTVGLHTKEDRNQLSWKIVKAFLYAHDHYLEYMDWFMKADDDICIYITLDNLKWLLTNYNPDESTYFGKRFKHCRKQDYMTGGAGYVLSKE

SEQ ID NO: 5 - DNA, 1560 bp, HUMAN
  CDS (2)..(934)
agataatggacagaatcatctagaaggacaaatgaacttcaatgcagattctagccaacataaagatgagaacacagacattgctgaaaacctctatcagaaagttagaattctttgctgggttatgaccggccctcaaaacctagagaaaaaggccaaacacgtcaaagctacttgggcccagcgttgtaacaaagtgttgtttatgagttcagaagaaaataaagacttccctgctgtgggactgaaaaccaaagaaggcagagatcaactatactggaaaacaattaaagcttttcagtatgttcatgaacattatttacaagatgctgattggtttttgaaagcagatgatgacacgtatgtcatactagacaatttgaggtggcttctttcaaaatacgaccctgaagaacccatttactttgggagaagatttaagccttatgtaaagcagggctacatgagtggaggagcaggatatgtactaagcaaagaagccttgaaaagatttgttgatgcatttaaaacagacaagtgtacacatagttcctccattgaagacttagcactggggagatgcatggaaattatgaatgtagaagcaggagattccagagataccattggaaaagaaacttttcatccctttgtgccagaacaccatttaattaaaggttatctacctagaacgttttggtactggaattacaactattatcctcctgtagagggtcctggttgctgctctgatcttgcagtttcttttcactatgttgattctacaaccatgtatgagttagaatacctcgtttatcatcttcgtccatatggttatttatacagatatcaacctaccttacctgaacgtatactaaaggaaattagtcaagcaaacaaaaatgaagatacaaaagtgaagttaggaaatccttgaaagaaaatcatgaatgaacaaaggtaatatgtctagcactgcactgaaaaaggacttctgcatttctgacatagaacactggaatcccagtgaggaattctaagtgaacattccttatagaaacctttcacatgaatgactataaactgaagctttaaatgagctgtgaagtgtgttaaaatgtgttttgatacagtaatatataaatatgtctatatatatgaggaacttgtgttttttaaatggtggccaggtagaggaactagaaaagagattttgttgcctgttttctgaccatctgtgttattgtcactgagaaactaaaatagtaaatttactaaaactacactgcaccatgttagtaataaacagatctgccttaaagaaaagaaaattttagaaagaaatattgttgctcagtgttgttaatatagctcaagaattgagtttatatttgcagtatgctataaatgatacccccctaccacacccacacacacagtttttgtctaatgaaaatgttgctgtgattatttataattggtagtatttcttccagaagaagctaaaataagactggcacttaccctgaagtgcattaataaaaccacactttaaaatta

SEQ ID NO: 6 - protein, 311 aa, HUMAN
DNGQNHLEGQMNFNADSSQHKDENTDIAENLYQKVRILCWVMTGPQNLEKKAKHVKATWAQRCNKVLFMSSEENKDFPAVGLKTKEGRDQLYWKTIKAFQYVHEHYLQDADWFLKADDDTYVILDNLRWLLSKYDPEEPIYFGRRFKPYVKQGYMSGGAGYVLSKEALKRFVDAFKTDKCTHSSSIEDLALGRCMEIMNVEAGDSRDTIGKETFHPFVPEHHLIKGYLPRTFWYWNYNYYPPVEGPGCCSDLAVSFHYVDSTTMYELEYLVYHLRPYGYLYRYQPTLPERILKEISQANKNEDTKVKLGNP

(19) United States
(12) Patent Application Publication
(10) Pub. No.: US 2005/0096269 A1
(43) Pub. Date: May 5, 2005

(54) Protein Cluster II

(76) Inventor: Anneli Attersand, Bromma (SE)

Correspondence Address: PFIZER INC., PATENT DEPARTMENT, MS8260-1611, EASTERN POINT ROAD, GROTON, CT, 06340, US

(21) Appl. No.: 10/881,528

(22) Filed: Jun. 30, 2004

Related U.S. Application Data
(63) Continuation of application No. 10/029,359, filed on Dec. 21, 2001, now abandoned.
(60) Provisional application No. 60/259,984, filed on Jan. 5, 2001.

(30) Foreign Application Priority Data
Dec. 22, 2000 (SE) 0004828-0

(51) Int. Cl. A61K 38/17; C07K 14/47; C07H 21/04

(52) U.S. Cl. 514/12; 530/350; 435/69.1; 435/320.1; 435/325; 536/23.5

(57) ABSTRACT

The present invention relates to the identification of a human gene family expressed in metabolically relevant tissues. The genes encode a group polypeptides referred to as “Protein Cluster II” which are predicted to be useful in the diagnosis of metabolic diseases, such as obesity and diabetes, as well as in the identification of agents useful in the treatment of the said diseases.

### TECHNICAL FIELD

The present invention relates to the identification of a human gene family expressed in metabolically relevant tissues. The genes encode a group polypeptides referred to as “Protein Cluster II” which are predicted to be useful in the diagnosis of metabolic diseases, such as obesity and diabetes, as well as in the identification of agents useful in the treatment of the said diseases.

### BACKGROUND ART

Metabolic diseases are defined as any of the diseases or disorders that disrupt normal metabolism. They may arise from nutritional deficiencies; in connection with diseases of the endocrine system, the liver, or the kidneys; or as a result of genetic defects. Metabolic diseases are conditions caused by an abnormality in one or more of the chemical reactions essential to producing energy, to regenerating cellular constituents, or to eliminating unneeded products arising from these processes. Depending on which metabolic pathway is involved, a single defective chemical reaction may produce consequences that are narrow, involving a single body function, or broad, affecting many organs and systems.

One of the major hormones that influence metabolism is insulin, which is synthesized in the beta cells of the islets of Langerhans of the pancreas. Insulin primarily regulates the direction of metabolism, shifting many processes toward the storage of substrates and away from their degradation. Insulin acts to increase the transport of glucose and amino acids as well as key minerals such as potassium, magnesium, and phosphate from the blood into cells. It also regulates a variety of enzymatic reactions within the cells, all of which have a common overall direction, namely the synthesis of large molecules from small units. A deficiency in the action of insulin (diabetes mellitus) causes severe impairment in (i) the storage of glucose in the form of glycogen and the oxidation of glucose for energy; (ii) the synthesis and storage of fat from fatty acids and their precursors and the completion of fatty-acid oxidation; and (iii) the synthesis of proteins from amino acids.

There are two varieties of diabetes. Type I is insulin-dependent diabetes mellitus (IDDM), for which insulin injection is required; it was formerly referred to as juvenile onset diabetes. In this type, insulin is not secreted by the pancreas and hence must be taken by injection. Type II, non-insulin-dependent diabetes mellitus (NIDDM) may be controlled by dietary restriction. It derives from insufficient pancreatic insulin secretion and tissue resistance to secreted insulin, which is complicated by subtle changes in the secretion of insulin by the beta cells. Despite their former classifications as juvenile or adult, either type can occur at any age; NIDDM, however, is the most common type, accounting for 90 percent of all diabetes. While the exact causes of diabetes remain obscure, it is evident that NIDDM is linked to heredity and obesity. There is clearly a genetic predisposition to NIDDM diabetes in those who become overweight or obese.

Obesity is usually defined in terms of the body mass index (BMI), i.e. weight (in kilograms) divided by the square of the height (in meters). Weight is regulated with great precision. Regulation of body weight is believed to occur not only in persons of normal weight but also among many obese persons, in whom obesity is attributed to an elevation in the set point around which weight is regulated. The determinants of obesity can be divided into genetic, environmental, and regulatory.

Recent discoveries have helped explain how genes may determine obesity and how they may influence the regulation of body weight. For example, mutations in the ob gene have led to massive obesity in mice. Cloning the ob gene led to the identification of leptin, a protein coded by this gene; leptin is produced in adipose tissue cells and acts to control body fat. The existence of leptin supports the idea that body weight is regulated, because leptin serves as a signal between adipose tissue and the areas of the brain that control energy metabolism, which influences body weight.

Metabolic diseases like diabetes and obesity are clinically and genetically heterogeneous disorders. Recent advances in molecular genetics have led to the recognition of genes involved in IDDM and in some subtypes of NIDDM, including maturity-onset diabetes of the young (MODY) (Velho & Froguel (1997) Diabetes Metab. 23 Suppl 2:34-37). However, several IDDM susceptibility genes have not yet been identified, and very little is known about genes contributing to common forms of NIDDM. Studies of candidate genes and of genes mapped in animal models of IDDM or NIDDM, as well as whole genome scanning of diabetic families from different populations, should allow the identification of most diabetes susceptibility genes and of the molecular targets for new potential drugs. The identification of genes involved in metabolic disorders will thus contribute to the development of novel predictive and therapeutic approaches.

### DESCRIPTION OF THE INVENTION

According to the present invention, a family of genes and encoded homologous proteins (hereinafter referred to as “Protein Cluster II”) has been identified. Consequently, the present invention provides an isolated nucleic acid molecule selected from:
(a) nucleic acid molecules comprising a nucleotide sequence as shown in SEQ ID NO: 1, or 3;
(b) nucleic acid molecules comprising a nucleotide sequence capable of hybridizing, under stringent hybridization conditions, to a nucleotide sequence complementary to the polypeptide coding region of a nucleic acid molecule as defined in (a); and
(c) nucleic acid molecules comprising a nucleic acid sequence which is degenerate as a result of the genetic code to a nucleotide sequence as defined in (a) or (b).

The nucleic acid molecules according to the present invention includes cDNA, chemically synthesized DNA, DNA isolated by PCR, genomic DNA, and combinations thereof. RNA transcribed from DNA is also encompassed by the present invention.

The term “stringent hybridization conditions” is known in the art from standard protocols (e.g. Ausubel et al., supra) and could be understood as e.g. hybridization to filter-bound DNA in 0.5 M NaHPO₄, 7% sodium dodecyl sulfate (SDS), 1 mM EDTA at +65° C., and washing in 0.1 ×SSC/0.1% SDS at +68° C.

In a preferred form of the invention, the said nucleic acid molecule has a nucleotide sequence identical with SEQ ID NOS: 1 or 3 of the Sequence Listing. However, the nucleic acid molecule according to the invention is not to be limited strictly to the sequence shown as SEQ ID NOS: 1 or 3. Rather the invention encompasses nucleic acid molecules carrying modifications like substitutions, small deletions, insertions or inversions, which nevertheless encode proteins having substantially the features of the Protein Cluster II polypeptide according to the invention. Included in the invention are consequently nucleic acid molecules, the nucleotide sequence of which is at least 90% homologous, preferably at least 95% homologous, with the nucleotide sequence shown as SEQ ID NOS: 1 or 3 in the Sequence Listing.

Included in the invention is also a nucleic acid molecule which nucleotide sequence is degenerate, because of the genetic code, to the nucleotide sequence shown as SEQ ID NO: 1 or 3. A sequential grouping of three nucleotides, a “codon”, codes for one amino acid. Since there are 64 possible codons, but only 20 natural amino acids, most amino acids are coded for by more than one codon. This natural “degeneracy”, or “redundancy”, of the genetic code is well known in the art. It will thus be appreciated that the nucleotide sequence shown in the Sequence Listing is only an example within a large but definite group of sequences which will encode the Protein Cluster II polypeptide.

The nucleic acid molecules according to the invention have numerous applications in techniques known to those skilled in the art of molecular biology. These techniques include their use as hybridization probes, for chromosome and gene mapping, in PCR technologies, in the production of sense or antisense nucleic acids, in screening for new therapeutic molecules, etc.

More specifically, the sequence information provided by the invention makes possible large-scale expression of the encoded polypeptides by techniques well known in the art. Nucleic acid molecules of the invention also permit identification and isolation of nucleic acid molecules encoding related polypeptides, such as human allelic variants and species homologues, by well-known techniques including Southern and/or Northern hybridization, and PCR. Knowledge of the sequence of a human DNA also makes possible, through use of Southern hybridization or PCR, the identification of genomic DNA sequences encoding the proteins in Cluster II, expression control regulatory sequences such as promoters, operators, enhancers, repressors, and the like. Nucleic acid molecules of the invention are also useful in hybridization assays to detect the capacity of cells to express the proteins in Cluster II. Nucleic acid molecules of the invention may also provide a basis for diagnostic methods useful for identifying a genetic alteration(s) in a locus that underlies a disease state or states, which information is useful both for diagnosis and for selection of therapeutic strategies.

In a further aspect, the invention provides an isolated polypeptide encoded by the nucleic acid molecule as defined above. In a preferred form, the said polypeptide has an amino acid sequence according to SEQ ID NO: 2 or 4 of the Sequence Listing. However, the polypeptide according to the invention is not to be limited strictly to a polypeptide with an amino acid sequence identical with SEQ ID NO: 2 or 4 in the Sequence Listing. Rather the invention encompasses polypeptides carrying modifications like substitutions, small deletions, insertions or inversions, which polypeptides nevertheless have substantially the features of the Protein Cluster II polypeptide. Included in the invention are consequently polypeptides, the amino acid sequence of which is at least 90% homologous, preferably at least 95% homologous, with the amino acid sequence shown as SEQ ID NO: 2 or 4 in the Sequence Listing.

In a further aspect, the invention provides a vector harboring the nucleic acid molecule as defined above. The said vector can e.g. be a replicable expression vector, which carries and is capable of mediating the expression of a DNA molecule according to the invention. In the present context the term “replicable” means that the vector is able to replicate in a given type of host cell into which is has been introduced. Examples of vectors are viruses such as bacteriophages, cosmids, plasmids and other recombination vectors. Nucleic acid molecules are inserted into vector genomes by methods well known in the art.

Included in the invention is also a cultured host cell harboring a vector according to the invention. Such a host cell can be a prokaryotic cell, a unicellular eukaryotic cell or a cell derived from a multicellular organism. The host cell can thus e.g. be a bacterial cell such as an E. coli cell; a cell from a yeast such as Saccharomyces cervisiae or Pichia pastoris, or a mammalian cell. The methods employed to effect introduction of the vector into the host cell are standard methods well known to a person familiar with recombinant DNA methods.

In yet another aspect, the invention provides a process for production of a polypeptide, comprising culturing a host cell, according to the invention, under conditions whereby said polypeptide is produced, and recovering said polypeptide. The medium used to grow the cells may be any conventional medium suitable for the purpose. A suitable vector may be any of the vectors described above, and an appropriate host cell may be any of the cell types listed above. The methods employed to construct the vector and effect introduction thereof into the host cell may be any methods known for such purposes within the field of recombinant DNA. The recombinant polypeptide expressed by the cells may be secreted, i.e. exported through the cell membrane, dependent on the type of cell and the composition of the vector.

In a further aspect, the invention provides a method for identifying an agent capable of modulating a nucleic acid molecule according to the invention, comprising
(i) providing a cell comprising the said nucleic acid molecule;
(ii) contacting said cell with a candidate agent; and
(iii) monitoring said cell for an effect that is not present in the absence of said candidate agent.

For screening purposes, appropriate host cells can be transformed with a vector having a reporter gene under the control of the nucleic acid molecule according to this invention.

The expression of the reporter gene can be measured in the presence or absence of an agent with known activity (i.e. a standard agent) or putative activity (i.e. a “test agent” or “candidate agent”). A change in the level of expression of the reporter gene in the presence of the test agent is compared with that effected by the standard agent. In this way, active agents are identified and their relative potency in this assay determined.

A transfection assay can be a particularly useful screening assay for identifying an effective agent. In a transfection assay, a nucleic acid containing a gene such as a reporter gene that is operably linked to a nucleic acid molecule according to the invention, is transfected into the desired cell type. A test level of reporter gene expression is assayed in the presence of a candidate agent and compared to a control level of expression. An effective agent is identified as an agent that results in a test level of expression that is different than a control level of reporter gene expression, which is the level of expression determined in the absence of the agent. Methods for transfecting cells and a variety of convenient reporter genes are well known in the art (see, for example, Goeddel (ed.), Methods Enzymol., Vol. 185, San Diego: Academic Press, Inc. (1990); see also Sambrook, supra).

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

Additional features of the invention will be apparent from the following Examples. Examples 1 to 3 are actual, while Examples 4 to 9 are prophetic.

### EXAMPLES

### Example 1

### Identification of Protein Clusters

A family of homologous proteins (hereinafter referred to as “Protein Cluster II”) was identified by an “all-versus-all” BLAST procedure using all Caenorhabditis elegans proteins in the Wormpep20 database release (http://www.sanger.ac.uk/Projects/C₁₃ elegans/wormpep/index.shtml). The Wormpep database contains the predicted proteins from the C. elegans genome sequencing project, carried out jointly by the Sanger Centre in Cambridge, UK and the Genome Sequencing Center in St. Louis, USA. A number of 18,940 proteins were retrieved from Wormpep20. The proteins were used in a Smith-Waterman clustering procedure to group together proteins of similarity (Smith T. F. & Waterman M. S. (1981) Identification of common molecular subsequences. J. Mol. Biol. 147(1): 195-197; Pearson W R. (1991) Searching protein sequence libraries: comparison of the sensitivity and selectivity of the Smith-Waterman and FASTA algorithms. Genomics 11:635-650; Olsen et al. (1999) Optimizing Smith-Waterman alignments. Pac Symp Biocomput.302-313). Completely annotated proteins were filtered out, whereby 10,130 proteins of unknown function could be grouped into 1,800 clusters.

The obtained sequence clusters were compared to the Drosophila melanogaster proteins contained in the database Flybase (Berkeley Drosophila Genome Project; http://www.frutfly.org), and annotated clusters were removed. Non-annotated protein clusters, conserved in both C. elegans and D. melanogaster, were saved to a worm/fly data set, which was used in a BLAST procedure (http://www.ncbi.nlm.nih.gov/Education/BLASTinfo/information3.html) against the Celera Human Genome Database (http://www.celera.com). Overlapping fragments were assembled to, as close as possible, full-length proteins using the PHRAP software, developed at the University of Washington (http://www.genome.washington.edu/UWGC/analysistools/phrap.htm). A group of homologous proteins (“Protein Cluster II”) with unknown function was chosen for further studies.

EST databases provided by the EMBL (http://www.embl.org/Services/index.html) were used to check whether the human proteins in Cluster II were expressed, in order to identify putative pseudogenes. One putative pseudogene was identified and excluded.

### Example 2

### Analyses of Protein Cluster II

(a) Alignment

The human part of Protein Cluster II comprises polypeptides encoded by the nucleic acid sequences shown as SEQ ID NOS: 1, 3 and 5. The sequence shown as SEQ ID NO: 5 was identified as the gene annotated as Homo sapiens corel UDP-galactose:N-acetylgalactosamine-alpha-R beta 1,3-galactosyltransferase (C1GALT1; GenBank Accession No. AF155582; see also International Patent Application No. WO 99/65712.)

An alignment of the human polypeptides included in Protein Cluster II (SEQ ID NOS: 2, 4, and 6), using the ClustalW multiple alignment software (Thompson et al. (1994) Nucleic Acid Research 22:4673-4680) is shown in Table I. The alignment showed a high degree of conservation in a distinct part of the protein cluster II, indicating the presence of a novel domain (see positions marked with stars in Table I).

The sequence shown as SEQ ID NO: 6 is identical to positions 53-363 of the polypeptide encoded by the C1GALT1 gene mentioned above.

(b) HMM-Pfam

A HMM-Pfam search was performed on the human family members. Pfam is a large collection of protein families and domains. Pfam contains multiple protein alignments and profile-HMMs (Profile Hidden Markov Models) of these families. Profile-HMMs can be used to do sensitive database searching using statistical descriptions of a sequence family's consensus. Pfam is available on the WWW at http://pfam.wustl.edu; http.//www.sanger.ac.uk/Software/Pfam; and http://www.cgr.ki.se/Pfam. The latest version (4.3) of Pfam contains 1815 families. These Pfam families match 63% of proteins in SWISS-PROT 37 and TrEMBL 9. For references to Pfam, see Bateman et al. (2000) The Pfam protein families database. Nucleic Acids Res. 28:263-266; Sonnhammer et al. (1998) Pfam: Multiple Sequence Alignments and HMM-Profiles of Protein Domains. Nucleic Acids Research, 26:322-325; Sonnhammer et al. (1997) Pfam. a Comprehensive Database of Protein Domain Families Based on Seed Alignments. Proteins 28:405-420.

The HMM-Pfam search indicated that no previously known domains could be identified in Protein Cluster II, with exception for a weak homology to Galactosyltransferase (Pfam Accession No. PFO 1762; see also Kolbinger et al. (1998) J. Biol. Chem. 273: 433-440).

A Pfam-B search revealed identity to the Pfam-B 7357 domain (Pfam Accession No. PB007357). Pfam-B domains are generated automatically from an alignment taken from the database ProDom 2000.1 (http://www.lunix.toulouse.inra.fr/prodom) subtracting sequence segments already covered by Pfam-A. The ProDom database has been designed as tool to help analyze domain arrangements of proteins and protein families (Corpet et al. (1999) Nucleic Acid Research 27: 263-267). Pfam-B domains are curated manually at the Sanger Centre, UK, to become Pfam-A domains.

(c) TM-HMM

The human proteins in Cluster II were analyzed using the TM-HMM tool available e.g. at http://www.cbs.dtu.dk/services/TMHMM-1.0. TM-HMM is a method to model and predict the location and orientation of alpha helices in membrane-spanning proteins (Sonnhammer et al. (1998) A hidden Markov model for predicting transmembrane helices in protein sequences. ISMB 6:175-182). No transmembrane regions were identified.

(d) Analysis of Non-Human Orthologs

The Caenorhabditis elegans genome includes eight genes encoding proteins within Protein Cluster II, of which the closest ancestor in evolution, a sequence included the C. elegans cosmid C38H2.2 (GenBank Accession No. Z35461) and annotated as UDP-galactose:N-acetylgalactosamine-alpha-R beta 1,3-galactosyltransferase mRNA (GenBank Accession No. AF269063) is 55%, 54%, and 42% identical to the three identified human proteins shown as SEQ ID NOS: 2, 4 and 6, respectively. (See also: Genome sequence of the nematode C. elegans: a platform for investigating biology; The C. elegans Sequencing Consortium. Science (1998) 282:2012-2018. Published errata appear in Science (1999) 283:35; 283:2103; and 285:1493.)

The Drosophila melanogaster genome comprises 10 genes belonging to Protein Cluster II, of which the closest relative “CG9520” (GenBank Accession No. AE003623; see also Adams et al. (2000) The genome sequence of Drosophila melanogaster; Science 287:2185-2195) is 42% identical to the human protein set.

No counterparts to Protein Cluster II in Saccharomyces cerevisiae were identified.

### Example 3

### Expression Analysis

The tissue distribution of the human genes was studied using the Incyte LifeSeq® database (http://www.incyte.com). The nucleic acid molecules shown as SEQ ID NO: 1, 3 and 5 were found to be expressed primarily in germ cells and in the nervous system. Therefore, the said nucleic acid molecules shown as SEQ ID NO: 1, 3 and 5 and the polypeptides shown as SEQ ID NO: 2, 4 and 6 are proposed to be useful for differential identification of the tissue(s) or cell types(s) present in a biological sample and for diagnosis of diseases and disorders, including disorders of the central nervous system.

### Example 4

### Multiple Tissue Northern Blotting

Multiple Tissue Northern blotting (MTN) is performed to make a more thorough analysis of the expression profiles of the proteins in Cluster II. Multiple Tissue Northern (MTN™) Blots (http:www.clontech.com/mtn) are pre-made Northern blots featuring Premium Poly A+ RNA from a variety of different human, mouse, or rat tissues. MTN Blots can be used to analyze size and relative abundance of transcripts in different tissues. MTN Blots can also be used to investigate gene families and alternate splice forms and to assess cross species homology.

### Example 5

### Expressing Profiling Using Microarrays

Microarrays consist of a highly ordered matrix of thousands of different DNA sequences that can be used to measure DNA and RNA variation in applications that include gene expression profiling, comparative genomics and genotyping (For recent reviews, see e.g.: Harrington et al. (2000) Monitoring gene expression using DNA microarrays. Curr. Opin. Microbiol. 3(3):285-291; or Duggan et al. (1999) Expression profiling using cDNA Microarrays. Nature Genetics Supplement 21:10-14).

The expression pattern of the proteins in Cluster II can be analyzed using GeneChip® expression arrays (http://www.afflymetrix.com/products/app₁₃ exp.html). Briefly, mRNAs are extracted from various tissues. They are reverse transcribed using a T7-tagged oligo-dT primer and double-stranded cDNAs are generated. These cDNAs are then amplified and labeled using In Vitro Transcription (IVT) with T7 RNA polymerase and biotinylated nucleotides. The populations of cRNAs obtained are purified and fragmented by heat to produce a distribution of RNA fragment sizes from approximately 35 to 200 bases. GeneChip® expression arrays are hybridized with the samples. The arrays are washed and stained. The cartridges are scanned using a confocal scanner and the images are analyzed with the GeneChip 3.1 software (Affymetrix).

### Example 6

### Identification of Polypeptides Binding to Protein Cluster II

In order to assay for proteins interacting with Protein Cluster II, the two-hybrid screening method can be used. The two-hybrid method, first described by Fields & Song (1989) Nature 340:245-247, is a yeast-based genetic assay to detect protein-protein interactions in vivo. The method enables not only identification of interacting proteins, but also results in the immediate availability of the cloned genes for these proteins.

The two-hybrid method can be used to determine if two known proteins (i.e. proteins for which the corresponding genes have been previously cloned) interact. Another important application of the two-hybrid method is to identify previously unknown proteins that interact with a target protein by screening a two-hybrid library. For reviews, see e.g.: Chien et al. (1991) The two-hybrid system: a method to identify and clone genes for proteins that interact with a protein of interest. Proc. Natl. Acad. Sci. U.S.A. 88:9578-9582; Bartel P L, Fields (1995) Analyzing protein-protein interactions using two-hybrid system. Methods Enzymol. 254:241-263; or Wallach et al. (1998) The yeast two-hybrid screening technique and its use in the study of protein-protein interactions in apoptosis. Curr. Opin. Immunol. 10(2): 131-136. See also http://www.clontech.com/matchmaker.

The two-hybrid method uses the restoration of transcriptional activation to indicate the interaction between two proteins. Central to this technique is the fact that many eukaryotic transcriptional activators consist of two physically discrete modular domains: the DNA-binding domain (DNA-BD) that binds to a specific promoter sequence and the activation domain (AD) that directs the RNA polymerase II complex to transcribe the gene downstream of the DNA binding site. The DNA-BD vector is used to generate a fusion of the DNA-BD and a bait protein X, and the AD vector is used to generate a fusion of the AD and another protein Y. An entire library of hybrids with the AD can also be constructed to search for new or unknown proteins that interact with the bait protein. When interaction occurs between the bait protein X and a candidate protein Y, the two functional domains, responsible for DNA binding and activation, are tethered, resulting in functional restoration of transcriptional activation. The two hybrids are cotransformed into a yeast host strain harboring reporter genes containing appropriate upstream binding sites; expression of the reporter genes then indicates interaction between a candidate protein and the target protein.

### Example 7

### Full-Length Cloning of Cluster II Genes

The polymerase chain reaction (PCR), which is a well known procedure for in vitro enzymatic amplification of a specific DNA segment, can be used for direct cloning of Protein Cluster II genes. Tissue cDNA can be amplified by PCR and cloned into an appropriate plasmid and sequenced. For reviews, see e.g. Hooft van Huijsduijnen (1998) PCR-assisted cDNA cloning: a guided tour of the minefield. Biotechniques 24:390-392; Lenstra (1995) The applications of the polymerase chain reaction in the life sciences. Cellular & Molecular Biology 41:603-614; or Rashtchian (1995) Novel methods for cloning and engineering genes using the polymerase chain reaction. Current Opinion in Biotechnology 6:30-36. Various methods for generating suitable ends to facilitate the direct cloning of PCR products are given e.g. in Ausubel et al. supra (section 15.7).

In an alternative approach to isolate a cDNA clone encoding a full length protein of Protein Cluster II, a DNA fragment corresponding to a nucleotide sequence selected from the group consisting of SEQ ID NO: 1, 3, 5 or 7, or a portion thereof, can be used as a probe for hybridization screening of a phage cDNA library. The DNA fragment is amplified by the polymerase chain reaction (PCR) method. The primers are preferably 10 to 25 nucleotides in length and are determined by procedures well known to those skilled in the art. A lambda phage library containing cDNAs cloned into lambda phage-vectors is plated on agar plates with E. coli host cells, and grown. Phage plaques are transferred to nylon membranes, which are hybridized with a DNA probe prepared as described above. Positive colonies are isolated from the plates. Plasmids containing cDNA are rescued from the isolated phages by standard methods. Plasmid DNA is isolated from the clones. The size of the insert is determined by digesting the plasmid with appropriate restriction enzymes. The sequence of the entire insert is determined by automated sequencing of the plasmids.

### Example 8

### Recombinant Expression of Proteins in Eukaryotic Host Cells

To produce proteins of Cluster II a polypeptide-encoding nucleic acid molecule is expressed in a suitable host cell using a suitable expression vector and standard genetic engineering techniques. For example, the polypeptide-encoding sequence is subcloned into a commercial expression vector and transfected into mammalian, e.g. Chinese Hamster Ovary (CHO), cells using a standard transfection reagent. Cells stably expressing a protein are selected. Optionally, the protein may be purified from the cells using standard chromatographic techniques. To facilitate purification, antisera is raised against one or more synthetic peptide sequences that correspond to portions of the amino acid sequence, and the antisera is used to affinity purify the protein.

### Example 9

### Determination of Gene Function

Methods are known in the art for elucidating the biological function or mode of action of individual genes. For instance, RNA interference (RNAi) offers a way of specifically and potently inactivating a cloned gene, and is proving a powerful tool for investigating gene function. For reviews, see e.g. Fire (1999) RNA-triggered gene silencing. Trends in Genetics 15:358-363; or Kuwabara & Coulson (2000) RNAi-prospectsfor a general technique for determining gene function. Parasitology Today 16:347-349. When double-stranded RNA (dsRNA) corresponding to a sense and antisense sequence of an endogenous mRNA is introduced into a cell, the cognate mRNA is degraded and the gene is silenced. This type of posttranscriptional gene silencing (PTGS) was first discovered in C. elegans (Fire et al., (1998) Nature 391:806-811). RNA interference has recently been used for targeting nearly 90% of predicted genes on C. elegans chromosome I (Fraser et al. (2000) Nature 408: 325-330) and 96% of predicted genes on C. elegans chromosome III (Gönczy et al. (2000) Nature 408:33 1-336).

TABLE I
Alignment of polypeptides in Protein Cluster II:

| | | |
|---|---|---|
| SEQ_ID_NO_4 | ------------------------------------------------------------ | |
| SEQ_ID_NO_6 | ------------------------------------------------------------ | |
| SEQ_ID_NO_2 | MTENSLSEMASKSWLNFLTFLYGSAIGFILFSQLLSILLGEEGDTQTNVLHNDPHARHSD | 60 |
| SEQ_ID_NO_4 | -----------------------NTGVTDKLYQKMKILCWIMTGPQNLEKKIRRIRDTWA | 37 |
| SEQ_ID_NO_6 | DNGQNHLEGQMNFNADSSQHKDENTDIAENLYQKVRILCWVMTGPQNLEKKAKHVKATWA | 60 |
| SEQ_ID_NO_2 | DNGQNHLGGQMNFNADSSQRKDENTEIAENLYXQVKILCWVMTGSQNLQKKAKHVKATWA | 120 |
| | ** ** **** *** *** ** *** | |
| SEQ_ID_NO_4 | QGCNKALFMSSKENKDFSTVGLHTKEDRNQLSWKIVKAFLYAHDHYLEYMDWFMKADDDI | 97 |
| SEQ_ID_NO_6 | QRCNKVLFMSSEENKDFPAVGLKTKEGRDQLYWKTIKAFQYVHEHYLEDADWFLKADDDT | 120 |
| SEQ_ID_NO_2 | QRCLKVFFMSSEENKDFRAVGLKTKAGRDELYWKTINLF--------------------- | 159 |
| | * * * **** ***** *** ** * * ** * | |
| SEQ_ID_NO_4 | CIYITLDNLKWLLTNYNPDESTYEGKRFKHCRKQDYMTGGAGYVLSKE------------ | 145 |
| SEQ_ID_NO_6 | --YVILDNLRWLLSKYDPEEPIYFGRRFKPYVKQGYMSGGAGYVLSKEALKREVDAEKTD | 178 |
| SEQ_ID_NO_2 | ------------------------------------------------------------ | |
| SEQ_ID_NO_4 | ------------------------------------------------------------ | |
| SEQ_ID_NO_6 | KCTHSSSIEDLALGRCMEIMNVEAGDSRDTIUKETEHPFVPEHHLIKGYLPRTEWYWNYN | 238 |
| SEQ_ID_NO_2 | ------------------------------------------------------------ | |
| SEQ_ID_NO_4 | ------------------------------------------------------------ | |
| SEQ_ID_NO_6 | YYPPVEGPGCCSDLAVSFHYVDSTTMYELEYLVYHLRPYGYLYRYQPTLPERILKEISQA | 298 |
| SEQ_ID_NO_2 | ------------------------------------------------------------ | |
| SEQ_ID_NO_4 | ------------- | |
| SEQ_ID_NO_6 | NKNEDTKVKLGNP | 311 |
| SEQ_ID_NO_2 | ------------- | |

“*” = identical or conserved residues in all sequences in the alignment.

1-8. (canceled).

9. An isolated nucleic acid molecule selected from: (a) nucleic acid molecules consisting of a nucleotide sequence as shown in SEQ ID NO: 1 or 3, or a nucleotide sequence which is at least 90% homologous with a nucleotide sequence as shown in SEQ ID NO: 1 or 3; (b) nucleic acid molecules consisting of a nucleotide sequence capable of hybridizing, along its full length, under stringent hybridization conditions, to a nucleotide sequence complementary to the polypeptide coding region of a nucleic acid molecule as defined in (a); and (c) nucleic acid molecules consisting of a nucleic acid sequence which is degenerate as a result of the genetic code to a nucleotide sequence as defined in (a) or (b).

10. A vector harboring the nucleic acid molecule according to claim 9.

11. A replicable expression vector which carries and is capable of mediating the expression of a nucleotide sequence according to claim 9.

12. A cultured host cell harboring a vector according to claim 10.

13. A process for production of a polypeptide, comprising culturing a host cell according to claim 12 under conditions whereby said polypeptide is produced, and recovering said polypeptide.

14. A cultured host cell harboring a vector according to claim 11.

15. A process for production of a polypeptide, comprising culturing a host cell according to claim 14 under conditions whereby said polypeptide is produced, and recovering said polypeptide.

16. An isolated nucleic acid molecule according to claim 9, selected from the group consisting of nucleic acid molecules consisting of a nucleotide sequence as shown in SEQ ID NO: 1 or 3, or a nucleotide sequence which is at least 90% homologous with a nucleotide sequence as shown in SEQ ID NO: 1 or 3.

17. An isolated nucleic acid molecule according to claim 9, selected from the group consisting of nucleic acid molecules consisting of a nucleotide sequence as shown in SEQ ID NO: 1 or 3.
